(19) United States
(12) Patent Application Publication
(10) Pub. No.: US 2015/0369813 A1
(43) Pub. Date: Dec. 24, 2015

(54) ANALYTICAL TEST STRIP WITH TIERED CAPILLARY CHAMBER

(71) Applicant: LifeScan Scotland Limited, Inverness (GB)

(72) Inventors: Scott SLOSS, Inverness (GB); Lynsey WHYTE, Newtonmore (GB); Antony SMITH, Dingwall (GB); David MCCOLL, Inverness (GB)

(21) Appl. No.: 14/312,991

(22) Filed: Jun. 24, 2014

(51) Int. Cl. G01N 33/66; G01N 27/327; B01L 3/00; G01N 33/80

(52) U.S. Cl. CPC G01N 33/66; G01N 33/80; G01N 27/3275; B01L 3/502715; B01L 2300/0645; B01L 2300/0838; B01L 2300/0887; B01L 2300/0861; B01L 2300/0825

(57) ABSTRACT

An analytical test strip for the determination of an analyte (such as glucose) in, or a characteristic of, a bodily fluid sample includes an electrically-insulating base layer, a first patterned spacer layer disposed on the electrically-insulating base layer, a second patterned spacer layer disposed on the first patterned spacer layer; and a top hydrophilic layer disposed on the second patterned spacer layer. In addition, the electrically-insulating base layer, the first and second patterned spacer layers and the top hydrophilic layer define a tiered capillary chamber(s) that has a first tiered capillary chamber portion defined in the first patterned spacer layer and a second tiered capillary chamber portion defined in the second patterned spacer layer. Moreover, the first tiered capillary chamber portion and the second tiered capillary chamber portion are in direct fluidic communication with one another.

### BACKGROUND OF THE INVENTION

1. Field of the Invention

The present invention relates, in general, to medical devices and, in particular, to analytical test strips and related methods.

2. Description of Related Art

The determination (e.g., detection and/or concentration measurement) of an analyte (such as glucose) in, or a characteristic (for example hematocrit) of, a fluid sample is of particular interest in the medical field. For example, it can be desirable to determine glucose, ketone bodies, cholesterol, lipoproteins, triglycerides, acetaminophen, hematocrit, and/or HbA1c concentrations in a sample of a bodily fluid such as urine, blood, plasma or interstitial fluid. Such determinations can be achieved using analytical test strips, based on, for example, visual, photometric or electrochemical techniques. Conventional electrochemical-based analytical test strips are described in, for example, U.S. Pat. Nos. 5,708,247, and 6,284,125, each of which is hereby incorporated in full by reference.

### BRIEF DESCRIPTION OF THE DRAWINGS

The accompanying drawings, which are incorporated herein and constitute part of this specification, illustrate presently preferred embodiments of the invention, and, together with the general description given above and the detailed description given below, serve to explain features of the invention, in which:

FIG. 1 is a simplified exploded perspective view of an electrochemical-based analytical test strip according to an embodiment of the present invention;

FIG. 2 is a simplified perspective view of the electrochemical-based analytical test strip of FIG. 1;

FIG. 3 is a simplified top overlay view of a portion of the electrochemical-based analytical test strip of FIG. 1;

FIG. 4 is a simplified cross-sectional view of a portion of the electrochemical-based analytical test strip of FIG. 3 taken along line X-X of FIG. 3 and includes a depiction of various adhesive layers (not to scale) that were omitted from FIGS. 1, 2, and 3 for clarity;

FIG. 5 is a simplified cross-sectional view of a portion of the electrochemical-based analytical test strip of FIG. 3 taken along line Y-Y of FIG. 3 and includes a depiction of various adhesive layers (not to scale) that were omitted from FIGS. 1, 2, and 3 for clarity;

FIG. 6 is a simplified cross-sectional view of a portion of an analytical test strip according to an embodiment of the present invention;

FIG. 7A is a simplified top overlay view of a portion of a further analytical test strip according to an embodiment of the present invention;

FIGS. 7B-7E are a series of aligned simplified top views of various layers of the analytical test strip of FIG. 7A;

FIG. 8A is a simplified top overlay view of a portion of another analytical test strip according to an embodiment of the present invention;

FIGS. 8B-8E are a series of aligned simplified top views of various layers of the analytical test strip of FIG. 8A;

FIG. 9A is a simplified top overlay view of a portion of an additional analytical test strip according to an embodiment of the present invention

FIGS. 9B-9E are a series of aligned simplified top views of various layers of the analytical test strip of FIG. 9A;

FIG. 10A is a simplified top overlay view of a portion yet another analytical test strip according to an embodiment of the present invention;

FIGS. 10B-10E are a series of aligned simplified top views of various layers of the analytical test strip of FIG. 10A;

FIG. 11A is a simplified top overlay view of a portion yet a further analytical test strip according to an embodiment of the present invention;

FIGS. 11B-11E are a series of aligned simplified top views of various layers of the analytical test strip of FIG. 11A; and

FIG. 12 is a flow diagram depicting stages in a method for determining an analyte in a bodily fluid sample according to an embodiment of the present invention.

### DETAILED DESCRIPTION OF ILLUSTRATIVE EMBODIMENTS

The following detailed description should be read with reference to the drawings, in which like elements in different drawings are identically numbered. The drawings, which are not necessarily to scale, depict exemplary embodiments for the purpose of explanation only and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein.

In general, analytical test strips for the determination of an analyte (such as glucose) in a bodily fluid sample (for example, a whole blood sample) and/or a characteristic of the bodily fluid sample (for example, hematocrit) according to embodiments of the present invention include an electrically-insulating base layer, a first patterned spacer layer disposed on the electrically-insulating base layer, a second patterned spacer layer disposed on the first patterned spacer layer; and a top hydrophilic layer disposed on the second patterned spacer layer. In addition, the electrically insulating base layer, first patterned spacer layer, second patterned spacer layer and top hydrophilic layer define at least one tiered capillary chamber with the at least one tiered capillary chamber having a first tiered capillary chamber portion defined in the first patterned spacer layer and a second tiered capillary chamber portion defined in the second patterned spacer layer. Moreover, the first tiered capillary chamber portion and the second tiered capillary chamber portion are in direct fluidic communication with one another.

As used herein, the term “tiered” refers to an entity (such as a capillary chamber) that includes two (or more) capillary chamber portions one atop and overlapping the other. In other words, the tiered capillary chamber portions are arranged in layers such that one is at a higher level (i.e., positioned higher along a vertical axis) than the other in a stepped manner.

Analytical test strips, including electrochemical-based analytical test strips, according to embodiments of the present invention are beneficial in that, for example, characteristics (such as size, volume, chamber length, dimensional aspect ratio, and surface hydrophilicity) of the first tiered capillary chamber portion can be predetermined and optimized independently from the characteristics of the second tiered capillary chamber portion. For example, one of the tiered capillary chamber portions can be optimized for bodily fluid sample application and conveyance of the applied bodily fluid sample to another tiered capillary chamber portion(s) while another of the tiered capillary chamber portions can be optimized for determining an analyte in, or characteristic of, the applied bodily fluid sample.

The tiered capillary chamber portion optimized for bodily fluid sample application and conveyance can have, for example, a width in the range of 1200 microns to 3000 microns, a height in the range of 50 microns to 250 microns and an upper surface hydrophilicity contact angle in the range of 8 degrees to 15 degrees. The tiered capillary chamber portion optimized for determining an analyte and/or characteristic of a bodily fluid sample can have, for example, a width in the range of 800 microns to 1200 microns, a height in the range of 100 microns to 150 microns and an upper surface hydrophilicity contact angle in the range of 10 degrees to 15 degrees.

Analytical test strips, including electrochemical-based analytical test strips according to embodiments of the present invention are also beneficial in that they are relatively inexpensive to manufacture using conventional layer patterning and lamination techniques including web-based manufacturing methods.

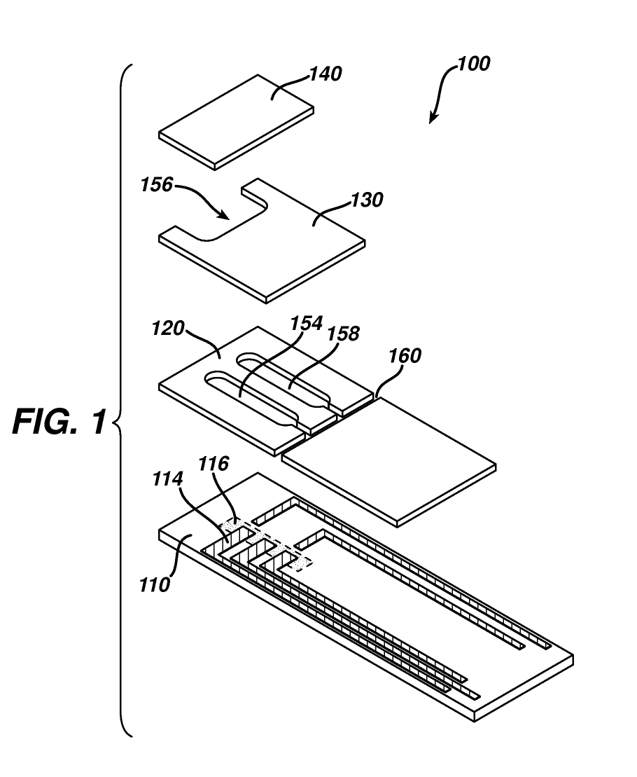

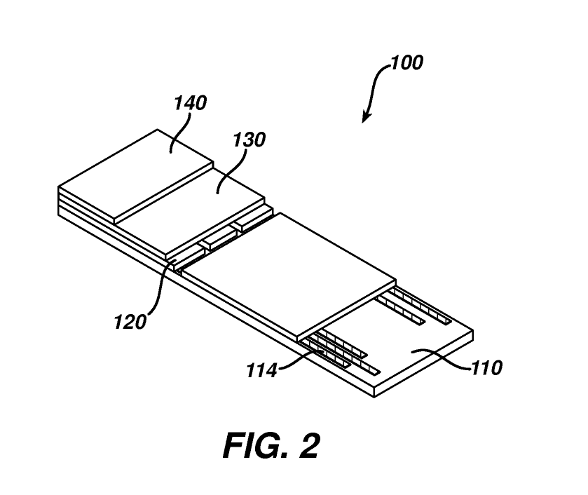

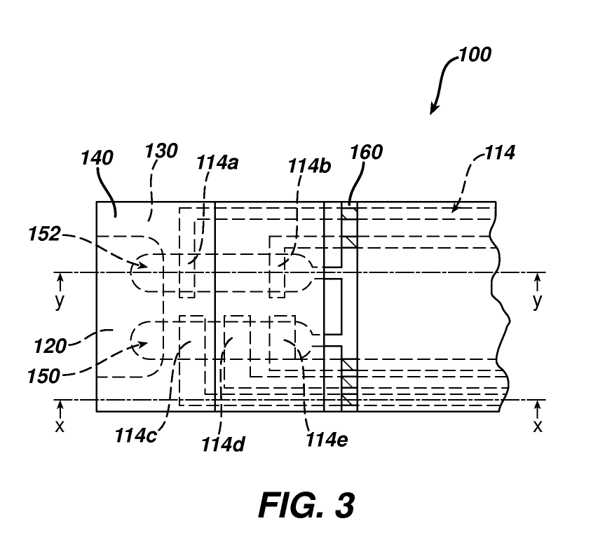

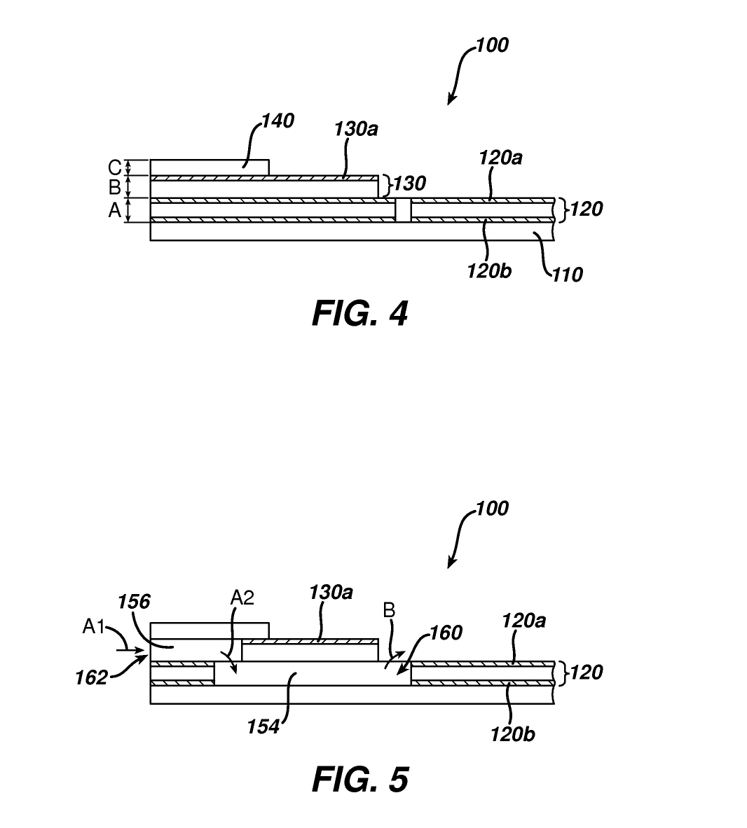

FIG. 1 is a simplified exploded perspective view of an analytical test strip (i.e., electrochemical-based analytical test strip 100) according to an embodiment of the present invention. FIG. 2 is a simplified perspective view of electrochemical-based analytical test strip 100. FIG. 3 is a simplified top overlay view of a portion of electrochemical-based analytical test strip 100 absent the reagent layer and top hydrophilic layer thereof. FIG. 4 is a simplified cross-sectional view of a portion of electrochemical-based analytical test strip 100 taken along line X-X of FIG. 3 and includes depictions of various adhesive sub-layers (not to scale) that are omitted from FIGS. 1, 2 and 3. FIG. 5 is a simplified cross-sectional view of a portion of electrochemical-based analytical test strip 100 taken along line Y-Y of FIG. 3 that also includes depictions of various adhesive sub-layers (not to scale) that were omitted from FIGS. 1, 2 and 3 for clarity.

Referring to FIGS. 1-5, electrochemical-based analytical test strip 100 for the determination of an analyte (such as glucose) in a bodily fluid sample (for example, a whole blood sample) and/or for the determination of a characteristic (for example hematocrit) of the bodily fluid sample includes an electrically-insulating base layer 110, a patterned conductor layer 114, a reagent layer 116, a first patterned spacer layer 120 disposed on electrically-insulating base layer 110, a second patterned spacer layer 130 disposed on first patterned spacer layer 120, and a top hydrophilic layer 140 disposed on second patterned spacer layer 130.

Electrically-insulating base layer 110, first patterned spacer layer 120, second patterned spacer layer 130 and top hydrophilic layer 140 define a first tiered capillary chamber 150 (see FIG. 3 in particular) and a second tiered capillary chamber 152 (see FIG. 3 in particular). First tiered capillary chamber 150 has a first tiered capillary chamber portion 154 defined in the first patterned spacer layer 120 and a second tiered capillary chamber portion 156 defined in second patterned spacer layer 130. Second tiered capillary chamber 152 also includes second tiered capillary chamber portion 156 defined in second patterned spacer layer 130 but in combination with a third tiered capillary chamber portion 158 defined in first patterned spacer layer 120. In other words, second tiered capillary chamber portion 156 is shared (i.e., in direct fluidic communication with) by both first tiered capillary chamber 150 and second tiered capillary chamber 152.

In the embodiment of FIGS. 1-5, first patterned spacer layer 120 is patterned to provide air vent 160. Moreover, second patterned spacer layer 130 is patterned to provide electrochemical-based analytical test strip 100 with a sample application opening 162 (see FIG. 5). Sample application opening 162 can have, for example, a width of 3 mm.

The first tiered capillary chamber portion 154 and the second tiered capillary chamber portion 156 are in direct fluidic communication (as indicated by, for example, arrows in FIG. 5 depicting bodily fluid sample application to sample application opening 162 of second tiered capillary chamber portion 156 (arrow A1 of FIG. 5) and bodily fluid sample flow (conveyance) from second tiered capillary chamber portion 156 to first tiered capillary chamber portion 154 (arrow A2 of FIG. 5). Arrow B in FIG. 5 indicates the movement of air from first tiered capillary chamber portion 154 through air vent 160.

Electrically-insulating base layer 110 can be any suitable electrically-insulating base layer known to one skilled in the art including, for example, a nylon substrate, polycarbonate substrate, a polyimide substrate, a polyvinyl chloride substrate, a polyethylene substrate, a polypropylene substrate, a glycolated polyester (PETG) substrate, or a polyester substrate. The electrically-insulating base layer can have any suitable dimensions.

Electrically-insulating base layer 110 provides structure to electrochemical-based analytical test strip 100 for ease of handling and also serves as a base for the application (e.g., printing or deposition) of subsequent layers (e.g., a patterned conductor layer).

Patterned conductor layer 114, including electrodes 114a, 114b, 114c, 114d and 114e thereof (see FIG. 3 in particular), of electrochemical-based analytical test strip 100 can be formed of any suitable conductive material including, for example, gold, palladium, platinum, indium, titanium-palladium alloys and electrically conducting carbon-based materials including carbon inks. It should be noted that patterned conductor layers employed in electrochemical-based analytical test strips according to embodiments of the present invention can take any suitable shape and be formed of any suitable materials including, for example, metal materials and conductive carbon materials.

Referring in particular to FIG. 4, the disposition of third electrode 114c, fourth electrode 114d and fifth electrode 114e and reagent layer 116 are such that electrochemical-based analytical test strip 100 is configured for the electrochemical determination of an analyte (for example, glucose) in a bodily fluid sample (such as a whole blood sample) that has filled first tiered capillary chamber portion 154. Moreover, first electrode 114a and second electrode 114b are disposed in third tiered capillary chamber portion 158 such that electrochemical-based analytical test strip 100 is configured for the determination of a characteristic (namely hematocrit) of a bodily fluid sample that has filled third tiered capillary chamber portion 158.

During use, a bodily fluid sample is applied to electrochemical-based analytical test strip 100 and transferred (conveyed) to both first tiered capillary chamber portion 154 and third tiered capillary chamber portion 158 via second tiered capillary chamber portion 156 by capillary action. Therefore, first and third tiered capillary chamber portions 154 and 158 are configured for electrochemical-based determinations and second tiered capillary chamber portion 156 is configured for conveying a bodily fluid sample to the first tiered capillary chamber portion

Reagent layer 116 can include any suitable enzymatic reagents, with the selection of enzymatic reagents being dependent on the analyte to be determined. For example, if glucose is to be determined in a blood sample, reagent layer 130 can include a glucose oxidase or glucose dehydrogenase along with other components necessary for functional operation. Reagent layer 116 can include, for example, glucose oxidase, tri-sodium citrate, citric acid, polyvinyl alcohol, hydroxyl ethyl cellulose, potassium ferrocyanide, antifoam, cabosil, PVPVA, and water. Further details regarding reagent layers, and electrochemical-based analytical test strips in general, are in U.S. Pat. Nos. 6,241,862 and 6,733,655, the contents of which are hereby fully incorporated by reference.

In the embodiment of FIGS. 1-5, first patterned spacer layer 120 is a double-sided adhesive tape. The adhesive sub-layers on either side are labeled 120a and 120b in FIGS. 4 and 5 but not otherwise depicted in the FIGS. Patterned spacer layer 120 can be, for example, a PET carrier tape coated with temperature activated adhesive on both sides. The temperature activated adhesive is used to bond first patterned spacer layer 120 to electrically-insulating base layer 110. An exemplary patterned spacer layer material is commercially available from Adhesive Research as ARCare 90503 and consists of a 50 micron PET carrier with approximately 22.5 um of adhesive coated on both sides.

In electrochemical-based analytical test strip 100, second patterned spacer layer 130 is a single-sided adhesive tape with an adhesive sub-layer layer 130a on the upper surface and has a bottom surface (which is the ceiling of first and third tiered capillary chamber portions 154 and 158) of predetermined hydrophilicity. Second patterned spacer layer 130 can be, for example, a PET carrier coated with a hydrophilic treatment on the bottom side (i.e., bottom surface) and a temperature activated adhesive on the top side. It should be noted that the hydrophilic nature of the underside of second patterned insulation layer 130 is a factor in the hydrophilicity of first and third tiered capillary chamber portions 154 and 158 but not a factor in the hydrophilicity of second tiered capillary chamber portion 156.

Top hydrophilic layer 140 can be formed of any suitable material and has a hydrophilic lower surface (which forms the ceiling of second tiered capillary chamber portion 156). Top hydrophilic layer 140 can be formed, for example, from a hydrophilic treated PET tape. Top hydrophilic layer 140 can be, for example, a clear film with hydrophilic properties that promote wetting and filling of electrochemical-based analytical test strip 100 by a bodily fluid sample (e.g., a whole blood sample). Such clear films are commercially available from, for example, 3M of Minneapolis, Minn. U.S.A. and Coveme (San Lazzaro di Savena, Italy). Top layer 140 can be, for example, a polyester film coated with a surfactant that provides a hydrophilic contact angle<10 degrees. Top hydrophilic layer 140 can also be a polypropylene film coated with a surfactant or other surface treatment, e.g., a MESA coating.

In FIGS. 4 and 5, height dimension A is in the range of, for example, approx. 90 μm to 150 μm, height dimension B is in the range of 100 μm to 300 μm and dimension C can be, for example, in the range of 50 μm to 100 μm. First and third tiered capillary chamber portions 154 and 158 have lengths of, for example, 4 mm and widths of, for example, 1 mm. However, analytical test strips and electrochemical-based analytical test strips according to the present invention can have any suitable dimensions are, therefore, not necessarily limited to the exemplary dimensions noted herein.

Although, for the purpose of explanation only, electrochemical-based analytical test strip 100 is depicted as including a total of five electrodes, embodiments of electrochemical-based analytical test strips, including embodiments of the present invention, can include any suitable number of electrodes.

Electrochemical-based analytical test strip 100 can be manufactured, for example, by the sequential aligned disposition of patterned conductor layer 114, reagent layer 116, first patterned spacer layer 120, second patterned spacer layer 130 and top hydrophilic layer 140 onto electrically-insulating base layer 110. Any suitable techniques known to one skilled in the art can be used to accomplish such sequential aligned formation, including, for example, screen printing, photolithography, photogravure, chemical vapour deposition and tape lamination techniques.

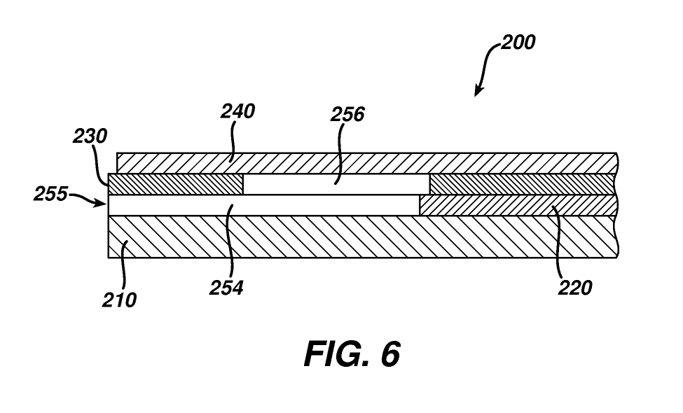

FIG. 6 is a simplified cross-sectional view of a portion of another analytical test strip 200 according to an embodiment of the present invention. Analytical test strip 200 includes an electrically-insulating base layer 210, a first patterned spacer layer 220 disposed on electrically-insulating base layer 210, second patterned spacer layer 230 disposed on first patterned spacer layer 220, and a top hydrophilic layer 240 disposed on second patterned spacer layer 230. For simplicity, air vent(s) of analytical test strip 200 are not depicted in the FIG.

In analytical test strip 200, first patterned spacer layer 220 is patterned to define a first tiered capillary chamber portion 254 and also a sample-application opening 255 in analytical test strip 200. Second patterned spacer layer 230 is patterned to define a second tiered capillary chamber portion 256. The combination of first tiered capillary chamber portion 254 and second tiered capillary chamber portion 256 taken together constitute a tiered capillary chamber of analytical test strip 200.

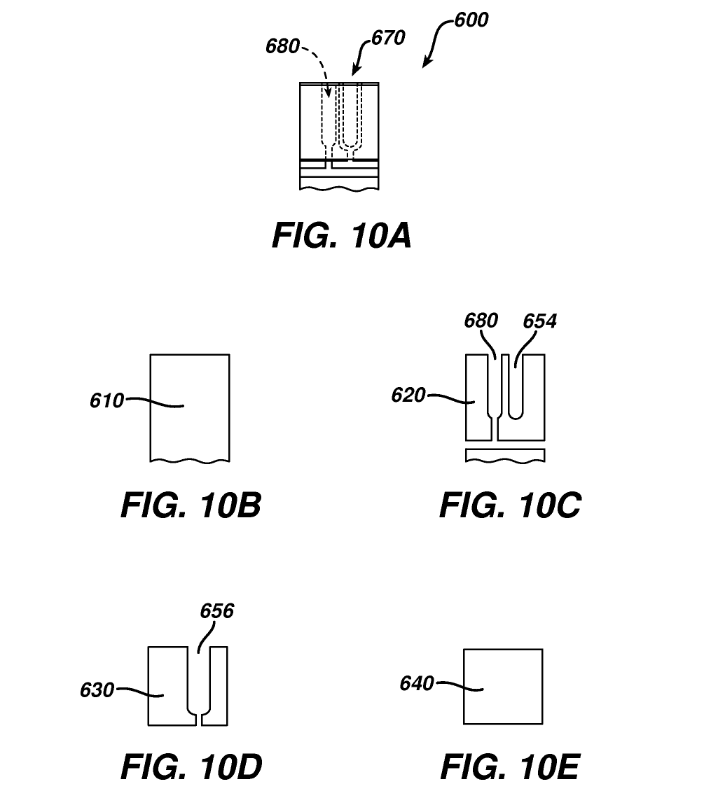

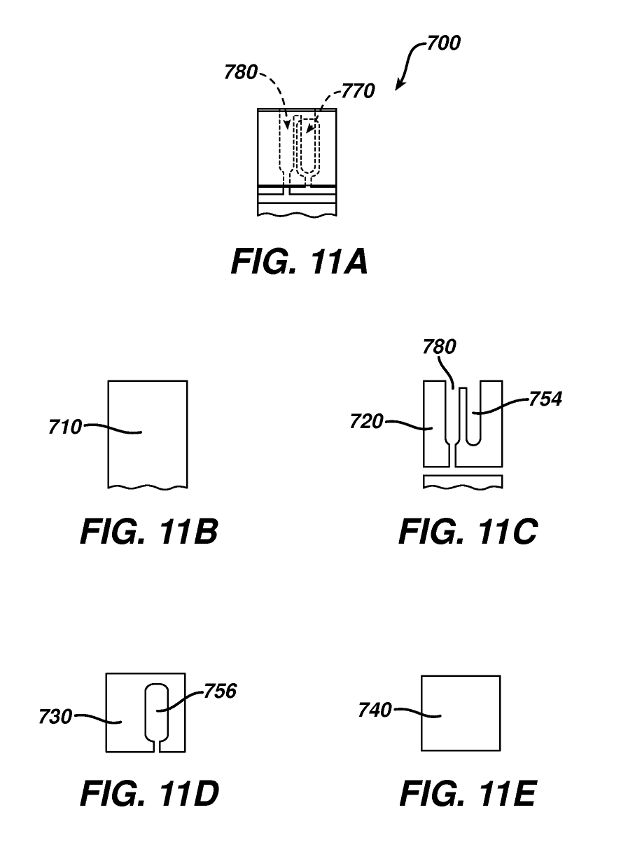

In electrochemical-based analytical test strip 100, the sample application opening was defined in the second patterned spacer layer. However, in analytical test strip 200, sample-application opening 255 is defined in the first patterned spacer layer. Moreover, it is noted that second tiered capillary portion 256 and first tiered capillary chamber portion 254 overlap to create a tiered capillary chamber with a height at the overlap that is greater than either tiered capillary chamber portion alone. Such an increased height can be beneficially employed in a differential measurement as explained further with respect to the embodiments of FIGS. 10A-10E and 11A-11E.

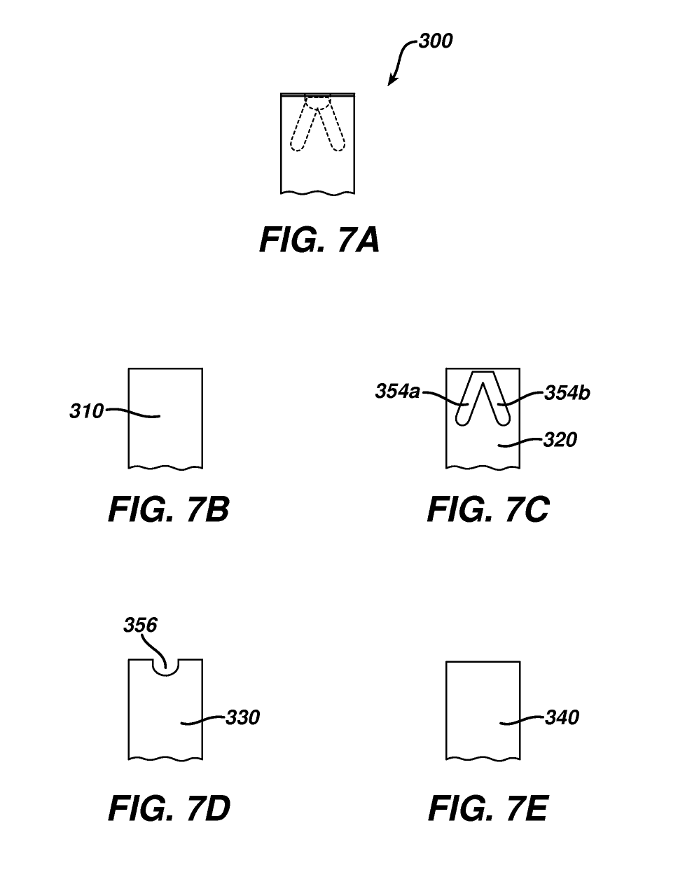

FIG. 7A is a simplified top overlay view of a portion of a further analytical test strip 300 according to an embodiment of the present invention. FIGS. 7B, 7C, 7D and 7E are a series of aligned simplified top views of various layers of analytical test strip 300. In FIGS. 7A-7E, like numerals indicate like elements in electrochemical-based analytical test strip 100. For clarity, vents(s) included in analytical test strip 300 are not depicted.

Referring to FIGS. 7A-7E, analytical test strip 300 includes an electrically-insulating base layer 310, a first patterned spacer layer 320 disposed on electrically-insulating base layer 310, second patterned spacer layer 330 disposed on first patterned spacer layer 320, and a top hydrophilic layer 340 disposed on second patterned spacer layer 330. For simplicity, air vent(s) of analytical test strip 300 are not depicted in the FIGS.

In analytical test strip 300, first patterned spacer layer 320 is patterned to define first tiered capillary chamber portions 354a and 354b. Second patterned spacer layer 330 is patterned to define a second tiered capillary chamber portion 356. The combination of first tiered capillary chamber portion 354a and second tiered capillary chamber portion 356 taken together constitute a tiered capillary chamber of analytical test strip 300, as does the combination of first tiered capillary chamber portion 354b and second tiered chamber portion 356. In the embodiment of FIGS. 7A-7E, first tiered capillary chamber portions 354a and 354b are configured in an intersecting V-shape.

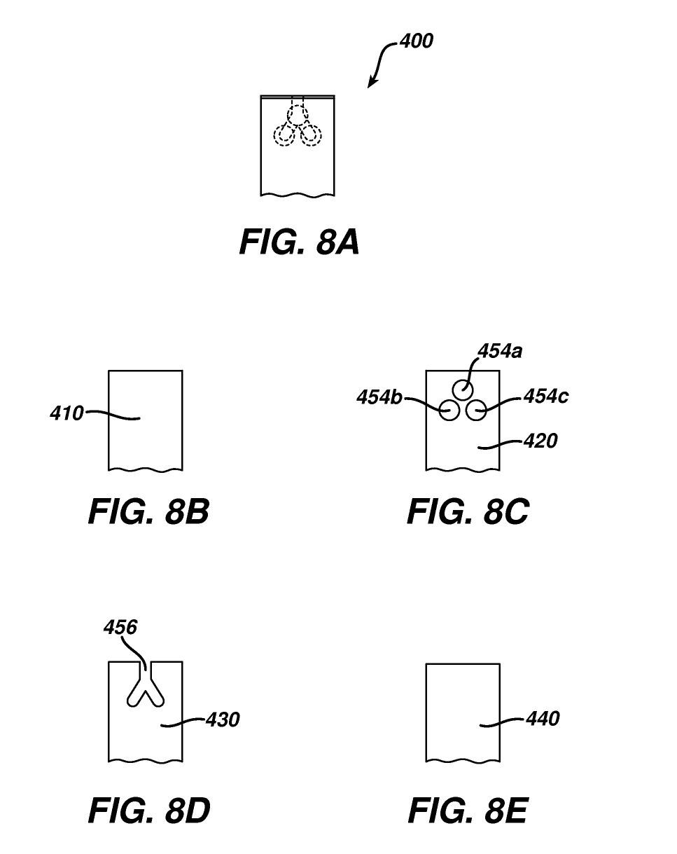

FIG. 8A is a simplified top overlay view of a portion of an analytical test strip 400 according to an embodiment of the present invention. FIGS. 8B, 8C, 8D and 8E are a series of aligned simplified top views of various layers of analytical test strip 400.

Referring to FIGS. 8A-8E, analytical test strip 400 includes an electrically-insulating base layer 410, a first patterned spacer layer 420 disposed on electrically-insulating base layer 410, second patterned spacer layer 430 disposed on first patterned spacer layer 420, and a top hydrophilic layer 440 disposed on second patterned spacer layer 430. For simplicity, air vent(s) of analytical test strip 400 are not depicted in the FIGS.

In analytical test strip 400, first patterned spacer layer 420 is patterned to define first tiered capillary chamber portions 454a, 454b, and 454c. Second patterned spacer layer 430 is patterned to define a second tiered capillary chamber portion 456. The combination of first tiered chamber portions 454a, 454b and 454c and second tiered chamber portion 456 taken together constitute three tiered capillary chambers of analytical test strip 400. First tiered capillary chamber portions 454a, 454b, and 454c are in circular configurations with each being in fluidic communication with second tiered sample chamber portion 456. The circular shape of first tiered capillary chamber portions 454a, 454b and 454c are beneficial in that (i) the circular shape can be readily manufactured using standard rotary punch tooling and (ii) the circular shape provides for efficient geometric packing of the first tiered capillary chamber portions.

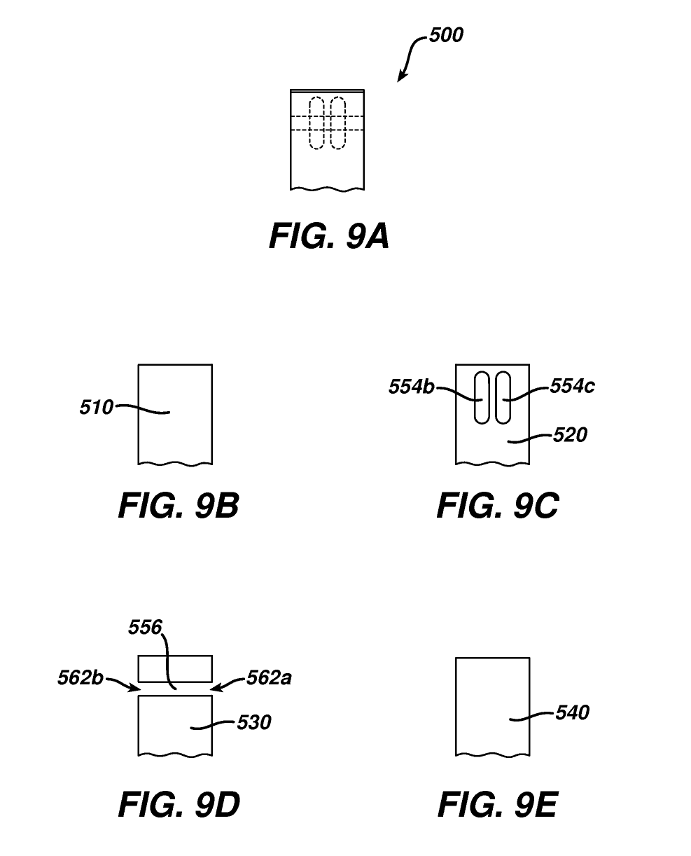

FIG. 9A is a simplified top overlay view of a portion of an additional analytical test strip 500 according to an embodiment of the present invention. FIGS. 9B, 9C, 9D and 9E are a series of aligned simplified top views of various layers of analytical test strip 500.

Referring to FIGS. 9A-9E, analytical test strip 500 includes an electrically-insulating base layer 510, a first patterned spacer layer 520 disposed on electrically-insulating base layer 510, second patterned spacer layer 530 disposed on first patterned spacer layer 520, and a top hydrophilic layer 540 disposed on second patterned spacer layer 530. For simplicity, air vent(s) of analytical test strip 500 are not depicted in the FIGS.

In analytical test strip 500, first patterned spacer layer 520 is patterned to define first tiered capillary chamber portions 554a and 554b. Second patterned spacer layer 530 is patterned to define a second tiered capillary chamber portion 556 that has two sample application openings 562a and 562b. The combination of first tiered capillary chamber portions 554a and 554b and second tiered capillary chamber portion 556 taken together constitute two tiered capillary chambers of analytical test strip 500.

FIG. 10A is a simplified top overlay view of a portion yet another analytical test strip 600 according to an embodiment of the present invention. FIGS. 10B, 10C, 10D and 10E are a series of aligned simplified top views of various layers of analytical test strip 600.

Referring to FIGS. 10A-10E, analytical test strip 600 includes an electrically-insulating base layer 610, a first patterned spacer layer 620 disposed on electrically-insulating base layer 610, a second patterned spacer layer 630 disposed on first patterned spacer layer 620, and a top hydrophilic layer 640 disposed on second patterned spacer layer 630.

In analytical test strip 600, first patterned spacer layer 620 is patterned to define a first tiered capillary chamber portions 654 and a non-tiered capillary chamber 680. Second patterned spacer layer 630 is patterned to define a second tiered capillary chamber portion 656. The combination of first tiered capillary chamber portion 654 and second tiered capillary chamber portion 656 taken together constitute a tiered capillary chamber 670 of analytical test strip 600.

Analytical test strip 600 includes both a tiered capillary chamber 670 and a non-tiered capillary chamber 680. The non-tiered capillary chamber is disposed entirely within first patterned layer 620. Therefore, the non-tiered sample capillary chamber is on a single-level and is also referred to herein as a single-level capillary sample chamber.

FIG. 11A is a simplified top overlay view of a portion yet a further analytical test strip 700 according to an embodiment of the present invention. FIGS. 11B, 11C, 11D and 11E are a series of aligned simplified top views of various layers of analytical test strip 700.

Referring to FIGS. 11A-11E, analytical test strip 700 includes an electrically-insulating base layer 710, a first patterned spacer layer 720 disposed on electrically-insulating base layer 710, second patterned spacer layer 730 disposed on first patterned spacer layer 720, and a top hydrophilic layer 740 disposed on second patterned spacer layer 730.

In analytical test strip 700, first patterned spacer layer 720 is patterned to define a first tiered capillary chamber portions 754 and a non-tiered capillary chamber 780. Second patterned spacer layer 730 is patterned to define a second tiered capillary chamber portion 756. The combination of first tiered capillary chamber portion 754 and second tiered capillary chamber portion 756 taken together constitute a tiered capillary chamber 770 of analytical test strip 700.

Analytical test strip 700 includes both a tiered capillary chamber 770 and a non-tiered capillary chamber 780. The non-tiered capillary chamber is disposed entirely within first patterned layer 720. Therefore, the non-tiered capillary chamber is on a single-level and is also referred to herein as a single-level capillary sample chamber.

With respect to the embodiments of FIGS. 10A-10E and 11A-11E, each of which includes both a tiered capillary chamber and a single-level (i.e., non-tiered) capillary chamber, it is envisioned that the tiered and single-level capillary chambers are configured such that they fill in a staged manner and/or at different rates. Such a staged fill and/or rate difference can be obtained, for example, by configuring the chambers with predetermined aspect ratios and/or hydrophilic surfaces and is also a result of the difference in their heights. Moreover, since one of the capillary chambers is tiered and one is non-tiered, it is hypothesized without being bound that there will be a difference in fill rate even if other factors are held constant. The difference in fill time or fill speed between the tiered and non-tiered capillary chamber is a differential measurement and thus tolerant of variation in strip manufacturing. It is envisioned that such as differential measurement can be used to determine various characteristics of the bodily fluid sample such as, for example, hematocrit.

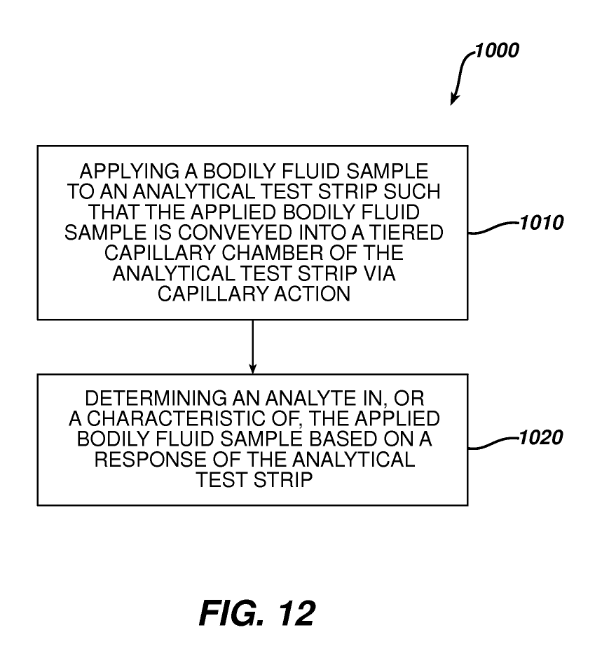

FIG. 12 is a flow diagram depicting stages in a method 1000 for determining an analyte (such as glucose) in a bodily fluid sample (for example, a whole blood sample) and/or a characteristic of the bodily fluid sample (e.g., hematocrit) according to an embodiment of the present invention. Method 1000 includes (see step 1010 of FIG. 12) applying a bodily fluid sample to an analytical test strip such that the applied bodily fluid sample is conveyed into at least one tiered capillary chamber of the analytical test strip via capillary action. In step 1010 the tiered capillary chamber has a first tiered capillary chamber portion defined in a first patterned spacer layer of the analytical test strip and a second tiered capillary chamber portion defined in a second patterned spacer layer of the analytical test strip. Moreover, the first tiered capillary chamber portion is in direct fluidic communication with the second tiered capillary chamber portion.

In the event that the applied bodily fluid sample is applied to the first tiered capillary chamber portion for conveyance to the second tiered capillary chamber portion, the capillary action for such conveyance can be optimized by using predetermined surface hydrophilicities of the first and second tiered capillary chamber portions, predetermined aspect ratios of the first and second tiered capillary chamber portions, and/or predetermined exposed edge configurations of the second tiered capillary chamber portion.

At step 1020 of method 1000, at least one of an analyte in, and a characteristic of, the applied bodily fluid sample, is determined based on a response of the analytical test strip.

Once apprised of the present disclosure, one skilled in the art will recognize that method 1000 can be readily modified to incorporate any of the techniques, benefits, features and characteristics of analytical test strips and electrochemical-based analytical test strips according to embodiments of the present invention and described herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that devices and methods within the scope of these claims and their equivalents be covered thereby.

What is claimed is:

1. An analytical test strip for the determination of an analyte in, or a characteristic of, a bodily fluid sample, the analytical test strip comprising:
an electrically-insulating base layer;
a first patterned spacer layer disposed on the electrically-insulating base layer;
a second patterned spacer layer disposed on the first patterned spacer layer; and
a top hydrophilic layer disposed on the second patterned spacer layer, and
wherein the electrically-insulating base layer, first patterned spacer layer, second patterned spacer layer and top hydrophilic layer define at least one tiered capillary chamber, the at least one tiered capillary chamber having:
a first tiered capillary chamber portion defined in the first patterned spacer layer; and
a second tiered capillary chamber portion defined in the second patterned spacer layer, and
wherein the first tiered capillary chamber portion and the second tiered capillary chamber portion are in direct fluidic communication.

2. The analytical test strip of claim 1 wherein the first tiered capillary chamber portion has a predetermined dimensional aspect ratio that differs from the predetermined dimensional aspect ratio of the second tiered capillary chamber portion.

3. The analytical test strip of claim 1 wherein at least one surface of the first tiered capillary chamber portion has a predetermined hydrophilicity, and
wherein at least one surface of the second tiered capillary chamber portion has a predetermined hydrophilicity, and
wherein the predetermined hydrophilicity of the surface of the second tiered capillary chamber portion is non-equivalent to the predetermined hydrophilicity of the surface of the first tiered capillary chamber portion.

4. The analytical test strip of claim 1 wherein an underside surface of the second patterned spacer layer forms a ceiling of the first tiered capillary chamber portion and the underside surface of the second patterned spacer layer has a predetermined hydrophilicity.

5. The analytical test strip of claim 1 further including:
a patterned conductor layer that includes a plurality of electrodes, and
a reagent layer, and
wherein the analytical test strip is configured as an electro-chemical based analytical test strip.

6. The analytical test strip of claim 1 wherein the first patterned spacer layer is a double-sided adhesive spacer layer.

7. The analytical test strip of claim 6 wherein the second patterned spacer layer is a single-sided adhesive spacer layer.

8. The analytical test strip of claim 1 wherein the analytical test strip further includes at least one non-tiered capillary chamber.

9. The analytical test strip of claim 1 wherein the at least one tiered capillary chamber is a plurality of tiered capillary chambers.

10. The analytical test strip of claim 1 wherein the first tiered capillary chamber portion is configured for conducting an electrochemical-based analyte determination and the second tiered capillary chamber portion is configured for conveying a bodily fluid sample to the first tiered capillary chamber portion.

11. The analytical test strip of claim 10 wherein the second tiered capillary chamber portion includes a sample application opening.

12. The analytical test strip of claim 1 wherein the second tiered capillary chamber portion is configured for conducting an electrochemical-based analyte determination and the first tiered capillary chamber portion is configured for conveying a bodily fluid sample to the first tiered capillary chamber portion.

13. The analytical test strip of claim 12 wherein the first tiered capillary chamber portion includes a sample application opening.

14. The analytical test strip of claim 1 wherein the analyte is glucose and the bodily fluid sample is blood.

15. The analytical test strip of claim 1 wherein the first tiered capillary portion has a height in the range of 100 microns to 150 microns and a hydrophilic contact angle in the range of 10 degrees to 15 degrees, and
wherein the second tiered capillary portion has a height in the range of 50 microns to 250 microns and a hydrophilic contact angle in the range of 8 degrees to 15 degrees. and the second tiered capillary portion

16. A method for determining at least one of an analyte in a bodily fluid sample and a characteristic of a bodily fluid sample, the method comprising:
applying a bodily fluid sample to an analytical test strip such that the applied bodily fluid sample is transported into at least one tiered capillary chamber of the analytical test strip via capillary action, the tiered capillary chamber having a first tiered capillary chamber portion defined in a first patterned spacer layer of the analytical test strip and a second tiered capillary chamber portion defined in a second patterned spacer layer of the analytical test strip, and wherein the first tiered capillary chamber portion is in direct fluidic communication with the second tiered capillary chamber portion; and
determining at least one of an analyte in, and a characteristic of, the applied bodily fluid sample based on a response of the analytical test strip.

17. The method of claim 16 wherein the response of the analytical test strip is an electrochemical-based analytical test strip.

18. The method of claim 16 wherein the response of the analytical test strip is a photometric response.

19. The method of claim 16 wherein the first tiered capillary chamber portion has a predetermined dimensional aspect ratio that differs from a predetermined dimensional aspect ratio of the second tiered capillary chamber portion.

20. The method of claim 16 wherein at least one surface of the first tiered capillary chamber portion has a predetermined hydrophilicity, and
wherein at least one surface of the second tiered capillary chamber portion has a predetermined hydrophilicity, and
wherein the predetermined hydrophilicity of the surface of the second tiered capillary chamber portion is non-equivalent to the predetermined hydrophilicity of the surface of the first tiered capillary chamber portion.

21. The method of claim 16 wherein the applied bodily fluid sample is conveyed into the at least one tiered capillary chamber and a non-tiered capillary chamber.

22. The method of claim 16 wherein the bodily fluid sample is whole blood.

23. The method of claim 16 wherein the analyte is glucose.

24. The method of claim 16 wherein the characteristic is hematocrit.

25. The method of claim 16 wherein the at least one tiered capillary chamber is a plurality of tiered capillary chambers.
